(19) United States
(12) Patent Application Publication
(10) Pub. No.: US 2014/0302614 A1
(43) Pub. Date: Oct. 9, 2014

(54) IMPEDANCE-BASED SENSOR FOR DETECTION OF CATALYST COKING IN FUEL REFORMING SYSTEMS

(71) Applicants: Jason M. Porter, Arvada, CO (US); Jeffrey L. Wheeler, Pullman, WA (US); Neal P. Sullivan, Louisville, CO (US)

(72) Inventors: Jason M. Porter, Arvada, CO (US); Jeffrey L. Wheeler, Pullman, WA (US); Neal P. Sullivan, Louisville, CO (US)

(21) Appl. No.: 14/245,341

(22) Filed: Apr. 4, 2014

Related U.S. Application Data
(60) Provisional application No. 61/808,306, filed on Apr. 4, 2013.

(51) Int. Cl. G01N 33/22; B01J 23/83; B01J 23/10; G01N 27/12

(52) U.S. Cl. CPC G01N 33/222; G01N 27/12; B01J 23/83; B01J 23/10
USPC 436/139; 422/82.02; 502/336; 502/303

(57) ABSTRACT

The present invention relates to a novel sensor for detecting the early stages of catalyst coking in fuel reforming systems and methods for making and using the same. The sensor may be manufactured by inkjet printing a colloidal suspension of ceramic powders to create thin (about 20 μm) catalytic and conductive elements of the sensor. The sensor may be used to determine the presence of coking conditions during processes at a level below the detection limit available using thermogravimetric analyzers (TGA) (<10 μg), thereby reducing catalyst coking in systems.

### CROSS REFERENCE TO RELATED APPLICATIONS

This application claims priority under 35 U.S.C. §119(e) to U.S. Provisional Application Ser. No. 61/808,306, filed on Apr. 4, 2013, which is incorporated herein in its entirety by reference.

### FIELD OF THE INVENTION

The present invention relates to a sensor for detecting the early stages of catalyst coking in systems and methods for making and using the sensors.

### SUMMARY

Hydrocarbon catalyst reforming is widely used for hydrogen production, fuel cell systems, and other industrial processes (collectively “hydrocarbon processes”). In these hydrocarbon processes, hydrocarbon based gases, for example methane, are mixed with steam and/or oxygen and exposed to a catalyst, such as nickel, to convert the hydrocarbon gas to a mixture of hydrogen, carbon monoxide and other trace species. Although widely used, a major limitation to these hydrocarbon processes is the formation of carbon on the catalyst surface, which is known as coking This carbon layer deactivates the catalyst, often causing permanent and expensive damage. Systems are currently designed to operate with excess steam and/or oxygen in order to avoid coking conditions but are inefficient. Furthermore, off-design conditions (e.g. an unexpected drop in oxygen) can lead to catalyst damage. Efficiency could be improved and off-design conditions avoided if a direct method of measuring coking was available.

Fouling by coke formation is a major source of catalyst deactivation in hydrocarbon reforming systems such as solid oxide fuel cells and steam methane reforming applications. Minor coking leads to a loss of catalyst surface area and decreases the efficiency of the reforming process. Severe coking may also lead to the mechanical failure of catalyst pellets as coke forms in the crevices of the catalyst material and lifts the catalyst metal from the pellet surface. Damage from coking may require the replacement of the catalyst from a hydrocarbon reforming system. These repairs require replacing expensive catalyst material and the shutdown of the reforming system for maintenance at significant expense.

A sensor which may detect coking in its early stages would allow for tighter control of stoichiometry in the fuel-reforming system. It is therefore desirable to have a sensor to monitor the catalyst for coking in real time. A sensor which could detect coke formation would allow a computer controlling the fuel stream or an operator to adjust the fuel mixture to prevent further coking before the catalyst is catastrophically damaged.

The present invention solves these and other problems by providing a sensor for early detection of catalyst coking in systems, and methods for making and using the sensors.

### BRIEF DESCRIPTION OF THE FIGURES

FIG. 1 illustrates a half bridge circuit and a sensor wired for a test;

FIG. 2 illustrates a schematic of the non-percolating nickel layer;

FIG. 3 illustrates SEM images of a catalyst surface;

FIG. 4 illustrates a sensor test set up;

FIG. 5 illustrates coking responses for different sensors;

FIG. 6 illustrates voltage response for a sensor;

FIG. 7 illustrates carbon-free sensor surfaces under FESEM;

FIG. 8 illustrates a sensor surface with carbon present under FESEM;

FIG. 9 illustrates nickel nodules;

FIG. 10 illustrates an unused and regenerated sensor;

FIG. 11 illustrates the circuit used to interface the sensor with a microcontroller; and

FIG. 12 illustrates the sensor's ability to automatically control a fuel stream.

### DETAILED DESCRIPTION

The present invention relates to a sensor to detect the presence of coke on a sensor and methods of making and using the same. The present invention also relates to an ink and the formation of the ink for use in the sensor.

An aspect of the invention is a sensor. The sensor comprises printed catalyst layers on a ceramic support with electrical connections. The catalyst layers may comprise two or more catalyst pads assembled on the substrate. The catalyst pads may be any shape, for example a circle, square, triangle, rectangle or the like. The catalyst pads may be any suitable size. In some embodiments, the length of the pads is between about 0.1 mm to about 10 mm, the width of each pad is between about 0.1 mm to about 10 mm. The thickness of the pad may be between about 5 μm to about 200 μm, in some embodiments about 20 μm thick. The pads may cover between about 1% to about 50% of the surface area of the substrate. The sensor operates by measuring small changes in the catalyst electrical conductivity caused by the formation of carbon on the catalyst layer. Furthermore, the sensor has a carbon sensitivity far beyond anything reported in the literature due to the carbon detection mechanism. The sensor may utilize an electrically resistive support material, including for example aluminum, silica and the like. In some embodiments, the sensor may comprise cobalt and/or iron. In some embodiments, the sensor may be non-percolating Ni-YSZ (yttria-stabilized zirconia) catalyst layer. This catalyst layer is similar to the catalysts found in fuel reforming systems except that it contains a lower concentration of nickel. In a typical supported catalyst, the metal catalyst particles contact each other and form an electronic conduction path through the catalyst material. In the non-percolating Ni-YSZ layer, the concentration of nickel is below about 20% by volume. In some embodiments, the concentration of nickel in the Ni-YSZ may be below about 15% by volume, below about 10% by volume, below about 5% by volume or below about 1% by volume. Other sensor embodiments may include lanthanum-doped strontium titanate (SLT). SLT may be used due to its electronic conductivity. SLT is electrically conductive and may provide electrical contacts for the resistance measurements needed. Other electronically conductive materials may be used in place of SLT. For example, platinum, gold, silver and/or lanthanum chromite. Furthermore, combinations of different sensor materials may be used. The substrate material may comprise a substrate that will have a similar coefficient of thermal expansion to the sensor material, which may be important during the manufacturing process (e.g. sintering) in order to prevent or decrease warping and cracking of the sensor material on the substrate. Furthermore, suitable substrate materials may be an inert high temperature material that is electrically insulating. One suitable material may be a partially stabilized zirconia (PSZ) disk (which may be green). Other suitable substrate materials include, but are not limited to, alumina and silica. The catalyst layers and/or other layers on the substrate may be connected to each other using a conductive material. In some embodiments, the conductive material is a silver paste. In some embodiments, the conductive material is a material doped with a conductive material, such as a material doped with silver particles.

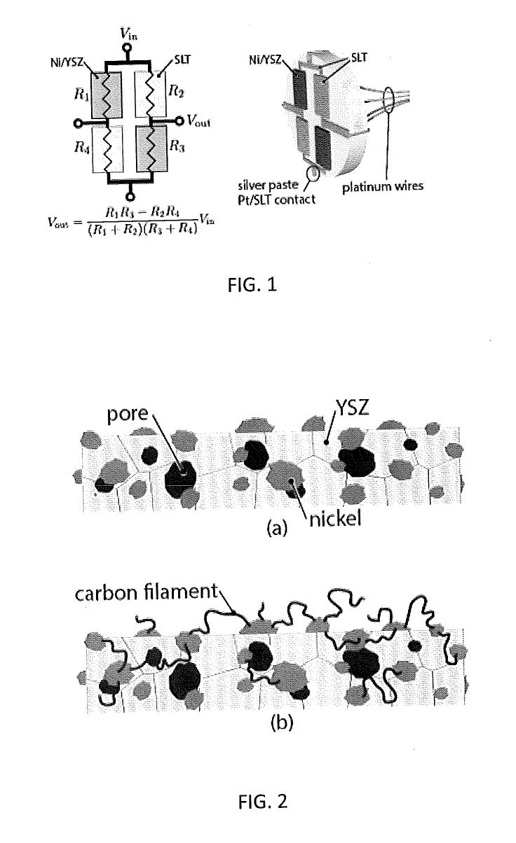

The sensor materials, including for example, SLT and NiO-YSZ, may be printed on the substrate in any suitable configuration. In some embodiments, the sensors may be printed in a pattern to form a half-bridge Wheatstone bridge circuit on a disk as illustrated in FIG. 1.

FIG. 2 illustrates a schematic of the non-percolating nickel layer. Nickel nodules may be dispersed throughout the YSZ grain structure and do not form electrical conduction paths through the layer. Pores may also be present in the layer, which allows carbon to form below the surface of the material. Since the nickel nodules do not form an electrically conductive path across the layer as illustrated in FIG. 2a, the electronic resistance of the material is high. As carbon forms in the catalyst layer as illustrated in FIG. 2b, carbon filaments grow from the nickel nodules and form electrical connections between nickel nodules. In addition to carbon filaments, carbon layers may also be formed. As connections form between nickel nodules, the electronic resistance of the material decreases.

An aspect of the present invention utilizes a sensor to detect the physical presence of coke on the sensor by measuring the electronic conductivity of carbon. A sensor is placed in a reactor. Coke formation is present when the electronic conductivity increases by between about 1% to about 50% of its initial electronic conductivity. A user or system may be notified once coke formation is beginning in the reactor so that appropriate action may be taken. Though not wanting to be bound by a particular theory, it is believed that this low concentration of nickel prevents the particles of nickel in the catalyst layer from connecting and forming a conduction path, resulting in very high electronic resistance. When coke begins to form on the nickel in the Ni-YSZ sensor catalyst layer, the nickel nodules are connected by carbon nanofibers and conduction through the catalyst layer increases. In some embodiments, the voltage drop may be calculated instead of the electronic resistance. A voltage drop between about 1% and about 20%, in some embodiments greater than about 4% compared to the initial voltage indicates the formation of coke. The classic Wheatstone bridge circuit may be used as the basis for the sensor design since the sensor detects coke formation by monitoring for a small change in electrical resistance. The Wheatstone bridge circuit may convert a small change in resistance into a voltage measurement which may be read easily with a voltmeter. Thus, by monitoring the change in resistance or voltage of the Ni-YSZ layer, it is possible to detect coking in its early stages. Once the sensor determines that coke is forming, a user may be notified (e.g. an alarm may sound, a message may be sent to a user) and/or the hydrocarbon process may cease or decrease the temperature in the hydrocarbon process to prevent coke formation.

An aspect of the present invention is a method for producing a sensor for detecting the presence of coking conditions. In some embodiments, an inkjet printer may be used to produce ceramics parts and may be used to print a variety of different materials including ceramic powders in liquid suspensions. The sensor may be produced using individual pieces produced utilizing an inkjet printer (i.e. the catalyst pads) or an inkjet printer may be used to produce the entire sensor. In some embodiments, the sensor was manufactured using a Fujifilm Dimatix DMP-2800 materials printer. Inkjet printers may be used to apply catalyst pad to a substrate. The inkjet printer may apply the ink that forms the catalyst pads to the substrate in any suitable shape or size. Advantageously, the inkjet printer may apply the pads to the substrate in a controlled manner and in any configuration at the suitable thickness. Following application of the catalyst pads, the sensor may be sintered in air between about 1000° C. to about 1600° C., in some embodiments about 1450° C., for between about 30 minutes to about 2 hours, in some embodiments about 1 hour. After sintering, the sensors may be placed in a chamber or furnace at between about 400° C. to about 1000° C., in some embodiments about 600° C. in an atmosphere containing between about 1 vol. % to about 10 vol. % hydrogen balanced with nitrogen, in some embodiments at about 3 vol. % hydrogen atmosphere balanced with nitrogen, for between about 12 hours to about 48 hours, in some embodiments about 24 hours to reduce the NiO to Ni.

An aspect of the present invention is the ink used in the inkjet printer to manufacture the sensors. In some embodiments, a suspension of the SLT and/or Ni-YSZ powders may be used. For example, in order to use the Dimatix printer to manufacture the sensors, it is necessary to make a suspension of the SLT and/or Ni-YSZ powders, which may be jetted from the printhead. The final viscosity of the suspension is critical to the success of jetting the suspension from the printhead. Thus, the solvent may be chosen in order to optimize the jetting, which may take into account the temperature of the suspension during jetting. In some embodiments, the solvent may be α-terpineol, water, alcohol, and/or ethylene glycol. The temperature dependent viscosity of α-terpineol may allow the Dimatix printer cartridge heater to hold the ink at an optimal temperature for jetting. In some embodiments, a dispersant may be used. Any suitable dispersant may be used. In some embodiments, the dispersant may be α-Terpineol. The dispersant may be ethylene glycol. Similarly, any suitable hyperdispersant may be used. By way of non-limiting example, the hyper dispersant may be Solsperse 13940. The dispersant may stabilize the suspension and prevent solids in the ink from settling. In some embodiments, a pore former may be used in the NiO-YSZ ink. The pore former may be an Esprix pore former from about 0.5 μm to about 3 μm. In some embodiments, starch and/or coal may be used as the pore former. In some embodiments, the pore former may be Esprix MX-150 about 1.5 μm PMMA particles. Table 1 illustrates suitable approximate compositions for an embodiment of NiO-YSZ Ink and SLT Ink.

TABLE 1

| Material | NiO-YSZ Ink (wt %) | SLT Ink (wt %) |
|---|---|---|
| α-Terpineol | 60-90 | 60-80 |
| NiO | 1-20 | — |
| YSZ | 1-20 | — |
| SLT | — | 10-40 |
| Dispersant | 0-10 | 0-10 |
| Pore former | 0-30 | — |

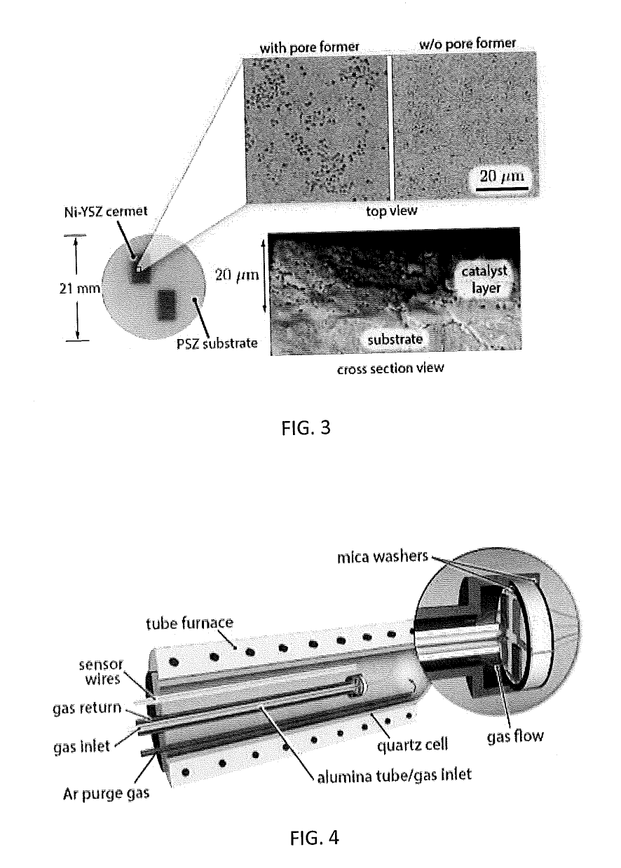

In some embodiments, the NiO-YSZ ink comprises about 77.6 wt % of α-Terpineol, about 3.8 wt % of the NiO, about 11.6 wt % of YSZ, about 5.7 wt % of a dispersant and about 1.3 wt % of a pore former. As the NiO content of the NiO-YSZ layer may be relatively low, pore former may be added to the formulation to promote porosity and high gas transport. Just as the low nickel concentration in the Ni-YSZ layer leads to low electronic conduction, the porosity created by the reduction of NiO to Ni is non-percolating, resulting in sub-optimal gas transport. A pore former may therefore be necessary to ensure adequate gas transport in the Ni-YSZ layer. The effect of adding pore former may be seen clearly under scanning electron microscope (SEM) as illustrated in FIG. 3. In some embodiments, the SLT ink comprises about 72.5 wt % of α-Terpineol, about 27 wt % of SLT and about 0.5 wt % of a dispersant.

### EXPERIMENTS

Current sensor testing consists of measuring changes in electrical potential across the sensor when exposed to coking environments. Tests to date have used high temperature ethylene as a coke precursor, followed by electron microscope imaging of carbon formation. Tests have also been completed under CO₂-methane reforming conditions, with the sensor response used to automatically shut down the reforming system when coking conditions were present.

### Experiment 1

The test stand used to evaluate the sensors consisted of a sensor holder, electrical test equipment, and a gas delivery system. The sensor holder consisted of a 1 in. Swagelok nut, which screwed onto a series of Swagelok adapters. The whole assembly was attached to the end of a 3/16 inch stainless steel tubing. The entire assembly was sealed inside a quartz tube for each test. The gas delivery system used three mass flow controllers to control the mixture of forming gas (about 3% hydrogen and about 97% nitrogen) and ethylene to control coking conditions. The gas was delivered to the sensor through an alumina tube which ran coaxially inside the 3/16 inch stainless steel tube of the sample holder. The stainless steel tube functioned as the exhaust of the test stand. Gas was also flowed around the outside of the sample holder in the quartz tube to purge any oxygen from the sensor holder. An illustration of the test stand set up is illustrated in FIG. 4.

The sensors were powered by a 5 V DC excitation voltage at the electrical contacts illustrated in FIG. 2. The 5 V DC excitation voltage was provided by a Fluke PM 2812 DC power supply. Voltage measurements were made by a Keithly 2420 sourcemeter programmed to operate as a voltmeter. Output voltage, temperature, and gas flow data are continuously measured and recorded using National Instruments data acquisition hardware and LabView software. Electrical connections were made by wrapping platinum wire over the edge of the sensor and using silver paste to connect the platinum to the SLT of the sensor. The wires were electronically insulated from the stainless steel sample holder by mica washers placed on the front and back of the sensors. The sensors were tested by heating the sample to about 600° C. under an about 0.2 SLPM flow of forming gas. The sensors were left at temperature overnight before testing to ensure the sensors had established a stable baseline voltage output. To produce coking conditions, about 0.05 SLPM of about 5% ethylene in argon was introduced into the flow of forming gas. The ethylene flow continued for between about 200 to about 2000 seconds while the voltage output of the sensor was monitored. Ethylene was selected as a coke precursor because it has been shown to be a significant source of coke formation.

### Results

### Coking Response

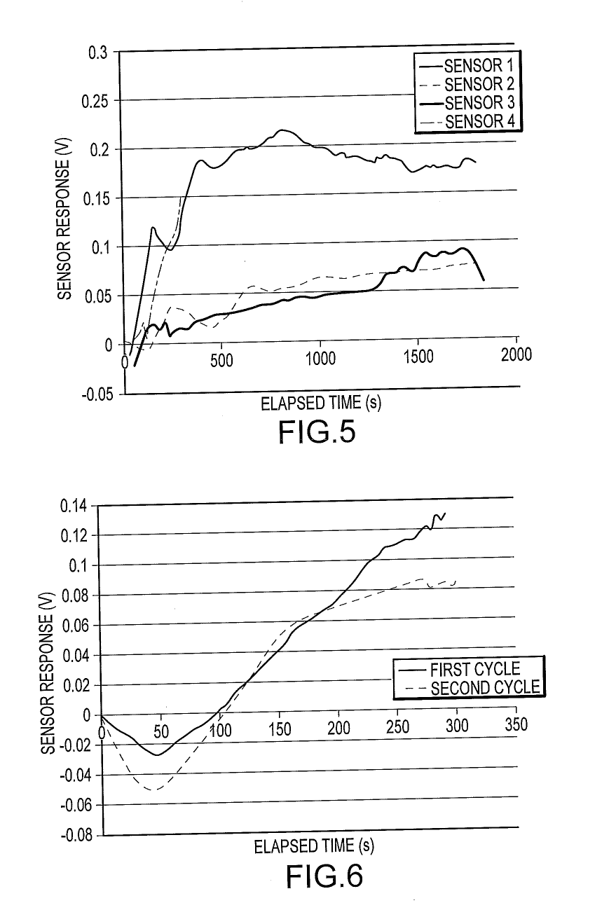

The sensors tested showed a strong response to coking as illustrated in FIG. 5. The sensor response is generally linear with a total voltage change of between about 5 mV and about 900 mV. The slope of the traces produced varied from about 0.05 mV/s to about 5 mV/s. The difference in the total voltage change and rate of voltage change is due to fabrication inconsistencies from sensor to sensor. While the Dimatix printer produced a consistent layer thickness and placement, the sensors must still be wired into the test stand by hand using silver paste, which introduces inconsistencies in the electrical connections from sensor to sensor. Also, certain batches of sensors had minor contamination issues during sintering. The contamination was a glassy substance which made small (about 0.1 mm maximum) droplets on the face of the sensor that introduced irregularities in the printed pattern of the sensor. Such irregularities would be expected to lower the signal-to-noise ratio of the sensor by adding resistance to the SLT connections on the sensor.

All sensors tested showed an increase in voltage which flattened out after about 200-2000 s. The voltage trace closely matches the growth behavior of carbon filaments observed in literature. The carbon filaments grow from a seed particle of the catalyst metal (nickel). Carbon bearing species are pyrolyzed on the particle surface and carbon diffuses into the metal. The carbon atoms diffuse through the metal and form layers of graphitic or amorphous carbon on the opposite side of the particle, resulting in a carbon filament. The filament grew in length at a linear rate until the catalyst particle at the tip of the filament was completely covered by a layer of carbon. Once the particle was encapsulated in carbon, the catalytic action of the particle was deactivated and the filament ceased to grow. The voltage trace of the sensor's coking response closely resembles the rate of carbon filament growth reported in the literature.

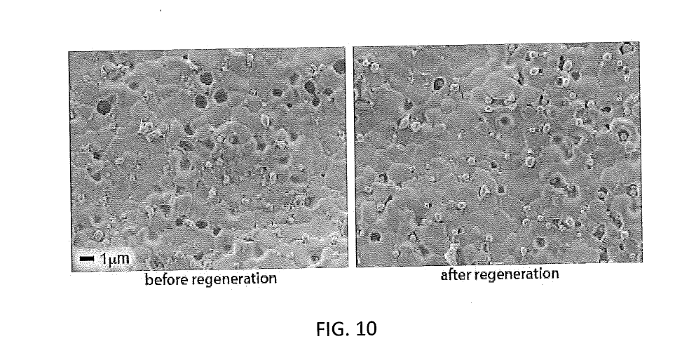

Tests were carried out to determine if the sensor may be regenerated after coking Coked sensors were exposed to wet forming gas (about 3% hydrogen, and about 97% nitrogen) at about 600° C. for about 24 hours. Water was introduced into the test stand by bubbling the gas through an ambient bubbler. The sensors were then subjected to a coking test identical to the original coking test. Sensors showed a decreased response to coking compared to the original test. Upon examining the microstructure of the regenerated sensor under field-effect scanning electron microscope (FESEM), nickel nodules were farther apart than uncoked samples, suggesting that some nickel was removed during the coking cycle as illustrated in FIG. 10. The missing nickel is likely the result of spalling caused by the lack of percolation of the nickel particles. Because the nickel particles do not percolate and do not bond to each other, it is believed that the nickel is bonding to YSZ which results in mechanically weak bonds. Undershoot was observed in some tests (as illustrated in FIG. 6). The cause of the undershoot shortly after the ethylene was introduced was possibly due to a gas-phase sensitivity.

### Closed-Loop Control Demonstration

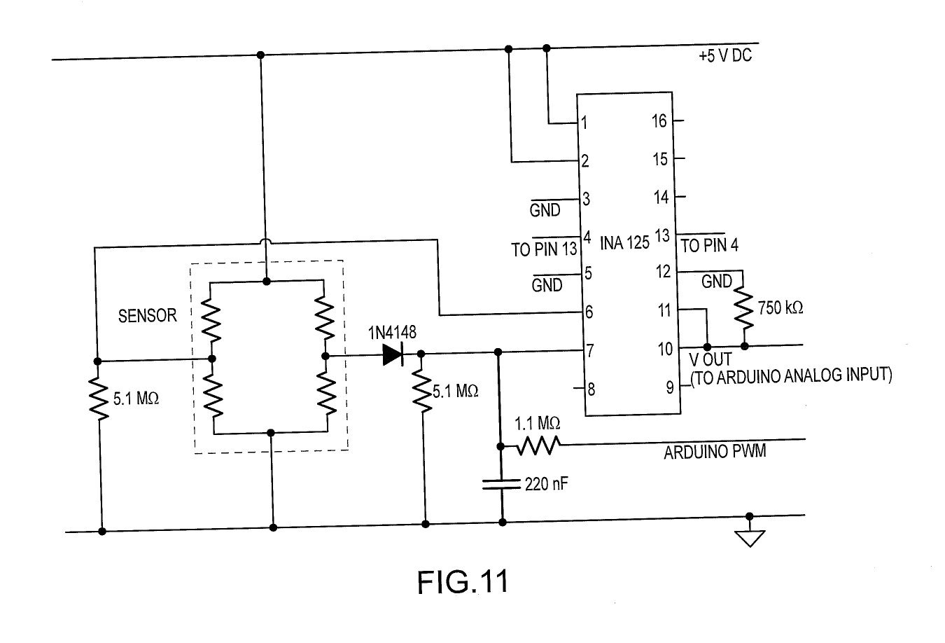

A test was carried out to demonstrate the sensor's ability to automatically control a fuel stream and respond to coking conditions. A custom circuit (FIG. 11) based on the Texas Instruments INA 125 instrumentation amplifier was constructed to interface the sensor to an Arduino UNO microcontroller. The custom circuit shown in FIG. 11 allowed the signal from the sensor to be read by an inexpensive microcontroller rather than a laboratory instrument. The signal read by the microcontroller was then interpreted by LabView. LabView was programmed to shut down the gas flow when the sensor detected coke formation.

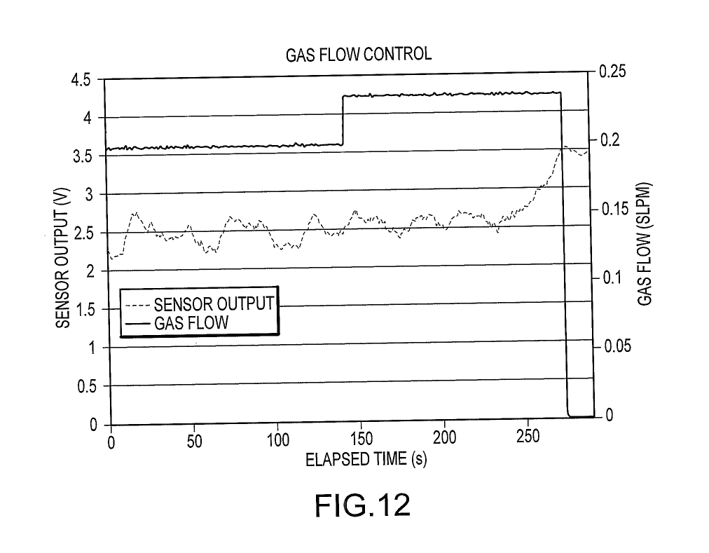

For the closed-loop control test, a gas mixture more representative of a real fuel stream was used. A mixture of methane and carbon dioxide was used to simulate a steam-methane reforming fuel stream. Methane-carbon dioxide mixtures have been shown to behave similarly to steam-methane mixtures in the conditions found in a fuel stream. At first, the sensor was exposed to an about 90% carbon dioxide, about 10% methane mixture at about 600° C. This mixture is known to be free of coke formation at about 600° C. While exposed to this mixture, the sensor displayed no coking response. Next the gas mixture was changed to about 78% carbon dioxide and about 22% methane. This mixture is known to produce coke at about 600° C. When the sensor was exposed to the coking mixture, a coking signal was detected and the gas flow was automatically shut down after about 130 s. FIG. 12 shows the results of this test.

### Examination of Coke Load

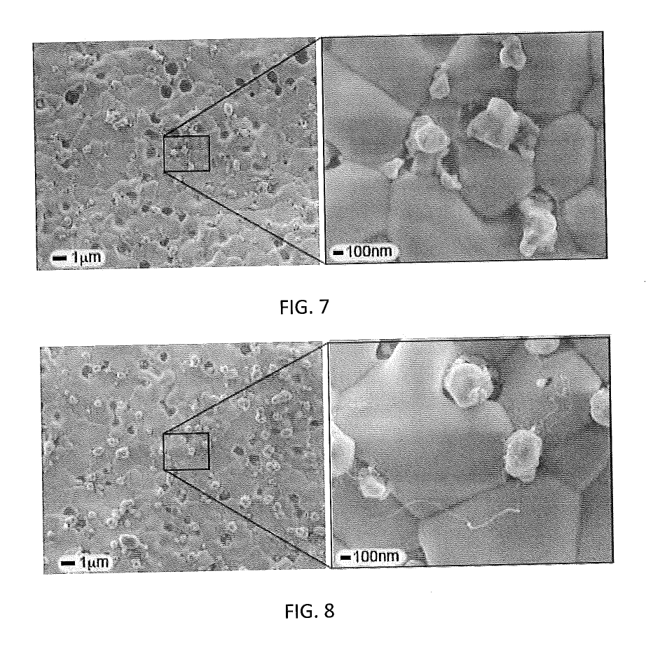

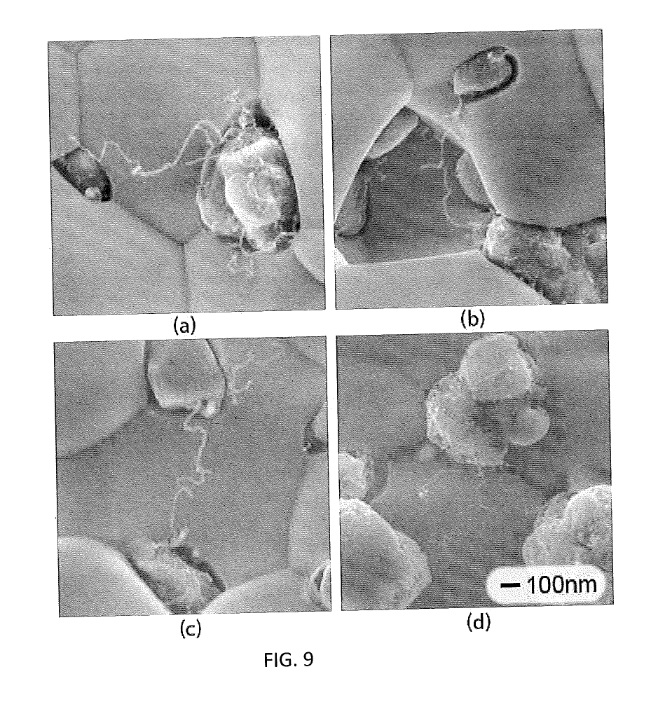

A FESEM was used to examine the surface of the coked sensor and obtained the image illustrated in FIGS. 8-9. The images from the FESEM show nickel nodules connected by filamentous carbon on the order of about 10-50 nm thick. Filamentous carbon has been observed to form under similar conditions on nickel catalysts. Since the carbon filaments are hundreds of times longer than their diameter, only a small mass of carbon is needed for a measurable decrease in sensor resistance. The result of the wire formation is that the non-percolating nickel surface becomes conductive when a small (less than about 10 μg) mass of coke forms on the surface of the sensor. FIG. 7 illustrates the surface of the non-percolating Ni-YSZ layer of a fresh, uncoked sensor. Nickel nodules may be seen dispersed throughout the YSZ grain structure. The nickel nodules have a rough appearance. In FIGS. 8 and 9 the surface of a coked sensor is shown. Carbon filaments may be seen connecting nickel nodules. The filaments on the sensor appear to have a small nickel particle at the tip which catalyzes filament growth. Similar filaments have been observed in coked catalysts. Filaments may be seen growing beneath the surface of the sensor through pores as illustrated in FIG. 9b. Filaments may also be seen growing towards each other and forming connections as illustrated in FIG. 9c. The nickel nodules have a smoother appearance in coked samples as illustrated in FIG. 8. Use of the microscopes energy-dispersive x-ray spectrometer (EDX) showed that there was a layer of carbon on the surface of the nickel nodules in addition to the carbon filaments. After regeneration, the sensor surface showed no connections between nickel nodules. The layer of carbon on the nodules was also gone. In addition to removing the carbon, some of the smaller nickel nodules were removed during the coking and regeneration cycle illustrated in FIG. 10.

An attempt was made to measure the mass of the coke load on a sensor using a Netzsch STA 409C thermogravimetric analyzer (TGA). The TGA removed the carbon by heating the sample to about 900° C. in a hydrogen-steam atmosphere. However, no measurable mass change was observed during the TGA tests. The resolution of the TGA was about 10 μg.

The foregoing description of the present invention has been presented for purposes of illustration and description. Furthermore, the description is not intended to limit the invention to the form disclosed herein. Consequently, variations and modifications commensurate with the above teachings, and the skill or knowledge of the relevant art, are within the scope of the present invention. The embodiment described hereinabove is further intended to explain the best mode known for practicing the invention and to enable others skilled in the art to utilize the invention in such, or other, embodiments and with various modifications required by the particular applications or uses of the present invention. It is intended that the appended claims be construed to include alternative embodiments to the extent permitted by the prior art.

1. A method for detecting the presence of coke, comprising:
placing a sensor in a reactor, wherein the sensor monitors an electronic conductivity;
monitoring the electronic conductivity to determine an initial electronic conductivity by monitoring a voltage; and
notifying a user when the electronic conductivity drops to between about 1% to about 50% of the initial electronic conductivity, wherein coke formation is present when the electronic conductivity is between about 1% to about 50% of the initial electronic conductivity.

2. The method of claim 1, wherein a user is notified when the sensor determines coke is forming to a predetermined level.

3. The method of claim 1, wherein carbon filaments are formed within the sensor.

4. A coke formation sensor for detecting the presence of coke on a catalyst surface, comprising:
a substrate;
at least one pad of a non-percolating Ni-YSZ catalyst layer attached to the substrate at a first location;
at least one pad of a second sensor material attached to the substrate at a second location; and
at least one electrical connection between the at least one pad of the non-percolating Ni-YSZ catalyst layer and the at least one pad of the second sensor material.

5. The sensor of claim 4, wherein the non-percolating Ni-YSZ catalyst layer comprises less than about 20% by volume of nickel.

6. The sensor of claim 4, wherein the substrate comprises partially stabilized zirconia.

7. The sensor of claim 4, wherein the at least one pad of the second sensor material is SLT.

8. The sensor of claim 4, wherein the at least one pad of the non-percolating Ni-YSZ catalyst layer comprises two pads, wherein a concentration of nickel in each of the two pads is less than about 20% by volume.

9. The sensor of claim 8, wherein the at least one pad of the non-percolating Ni-YSZ catalyst layer comprises pores.

10. The sensor of claim 4, wherein a material in the at least one electrical connection comprises a silver material.

11. The sensor of claim 4, wherein the at least one pad of the non-percolating Ni-YSZ catalyst layer and the at least one pad of the second sensor material comprise a Wheatstone bridge circuit.

12. A method for producing a coke formation sensor, comprising:
forming at least one catalyst pad on a substrate, wherein a material for the at least one catalyst pad comprises NiO-YSZ; and
forming at least one of a second pad of a second material on the substrate, wherein the at least one catalyst pad and the at least one second pad are in different locations on the coke formation sensor.

13. The method of claim 12, wherein the at least one catalyst pad and the at least one of the second pad are applied to the substrate with an inkjet printer.

14. The method of claim 12, wherein the NiO-YSZ is reduced to NiO-YSZ.

15. The method of claim 12, wherein a material for the at least one second pad is SLT.

16. The method of claim 12, further comprising:
sintering the coke formation sensor in air at a temperature between about 1000° C. and about 1600° C. for between about 30 minutes to about 2 hours to form a sintered coke formation sensor; and
reducing a material on the sintered coke formation sensor at a reducing temperature between about 400° C. to about 1000° C. in an atmosphere comprising between about 1% by volume to about 10% by volume of hydrogen and between about 90% by volume to about 99% by volume of nitrogen for between about 12 hours to about 48 hours to form a final coke formation sensor.

17. The method of claim 12, wherein a catalyst ink for use in an inkjet printer for the at least one catalyst pad comprises a suspension of NiO-YSZ comprising:
between about 60-90 wt % of α-Terpineol;
between about 1-20 wt % of NiO;
between about 1-20 wt % of YSZ;
between about 0-10 wt % of a dispersant; and
between about 0-30 wt % of a pore former.

18. The method of claim 17, wherein the suspension of NiO-YSZ comprises about 77.6 wt % of the α-Terpineol, about 3.8 wt % of the NiO, about 11.6 wt % of the YSZ, about 5.7 wt % of the dispersant and about 1.3 wt % of the pore former.

19. The method of claim 12, wherein an ink for the second material for use in an inkjet printer comprises a suspension of SLT comprising:
between about 60-80 wt % of α-Terpineol;
between about 10-40 wt % of SLT; and
between about 0-10 wt % of a dispersant.

20. The method of claim 19, wherein the suspension of SLT comprises about 72.5 wt % of the α-Terpineol, about 27 wt % of the SLT and about 0.5 wt % of the dispersant.
